(19) United States
(12) Patent Application Publication
(10) Pub. No.: US 2022/0124260 A1
(43) Pub. Date: Apr. 21, 2022

(54) Combined UV and Color Imaging System

(71) Applicant: X Development LLC, Mountain View, CA (US)

(72) Inventors: Eden Rephaeli, Oakland, CA (US); Guy Satat, Sunnyvale, CA (US); Abhinav Gupta, San Jose, CA (US); Hans Peter Brondmo, San Francisco, CA (US)

(21) Appl. No.: 17/354,908

(22) Filed: Jun. 22, 2021

Related U.S. Application Data
(60) Provisional application No. 63/093,692, filed on Oct. 19, 2020.

(51) Int. Cl. H04N 5/33; G06N 20/00; G06T 7/80; G06T 3/40

(52) U.S. Cl. CPC H04N 5/332; G06N 20/00; G06T 7/80; B25J 9/1697; G06T 2207/10024; G06T 2207/10064; G06T 3/4015

(57) ABSTRACT

A system includes a color camera configured to detect visible light and ultraviolet (UV) light, a UV illuminator, and a processor configured to perform operations. The operations include causing the UV illuminator to emit UV light towards a portion of an environment and receiving, from the color camera, a color image that represents the portion illuminated by the emitted UV light and by visible light incident thereon. The operations also include determining a first extent to which UV light is attenuated in connection with pixels of the color camera that have a first color and a second extent to which UV light is attenuated in connection with pixels of the color camera that have a second color. The operations further include generating a UV image based on the color image, the first extent, and the second extent, and identifying a feature of the environment by processing the UV image.

### CROSS-REFERENCE TO RELATED APPLICATIONS

This application claims priority to U.S. Provisional Patent Application No. 63/093,692, filed on Oct. 19, 2020, and titled “Combined UV Imaging and Sanitization,” which is hereby incorporated by reference as if fully set forth in this description.

### BACKGROUND

As technology advances, various types of robotic devices are being created for performing a variety of functions that may assist users. Robotic devices may be used for applications involving material handling, transportation, welding, assembly, and/or dispensing, among others. Over time, the manner in which these robotic systems operate is becoming more intelligent, efficient, and intuitive. As robotic systems become increasingly prevalent in numerous aspects of modern life, it is desirable for robotic systems to be efficient. Therefore, a demand for efficient robotic systems has helped open up a field of innovation in actuators, movement, sensing techniques, as well as component design and assembly.

### SUMMARY

A color camera, such as a red-green-blue (RGB) camera, may be used to determine an ultraviolet (UV) image of a scene. Specifically, the color camera may be used to capture a color image of a scene that is illuminated with UV light emitted by a UV illuminator, as well as visible light present in the environment. Thus, the color image may represent the scene illuminated by both the emitted UV light and by visible light incident on the scene at the time of image capture. The color camera may be configured to detect UV light in that (i) it may include components configured to transmit UV light to pixels of the camera and/or (ii) it might not include at least some components, such as UV filters, that would otherwise block the transmission of UV light to pixels of the camera. Nevertheless, the UV light may undergo some attenuation by optical components of the camera such as, for example, a color filter array of the camera. This extent of attenuation may be quantified on a per-color basis and/or on a per-pixel basis, and may be used to determine the UV image. In one example, the color image may be computationally decomposed into a visible light component and a separate UV light component, which may be used to determine the UV image. In another example, a second color image that represents the scene without UV light illumination may be subtracted from the color image, thus generating a difference image that includes the UV light component and lacks the visible light component, and thus allows for determination of the UV image.

In a first example embodiment, a system includes a color camera configured to detect visible light and UV light, a UV illuminator, a processor, and a non-transitory computer-readable storage medium having stored thereon instructions that, when executed by the processor, cause the processor to perform operations. The operations include causing the UV illuminator to emit UV light towards a portion of an environment, and receiving, from the color camera, a color image that represents the portion of the environment illuminated by the emitted UV light and by visible light incident on the portion of the environment. The operations also include determining (i) a first extent to which UV light is attenuated in connection with pixels of the color camera that have a first color and (ii) a second extent to which UV light is attenuated in connection with pixels of the color camera that have a second color. The operations additionally include generating a UV image based on the color image, the first extent, and the second extent. The operations further include identifying one or more features of the environment by processing the UV image.

In a second example embodiment, a method may include causing a UV illuminator to emit UV light towards a portion of an environment, and receiving, from a color camera configured to detect visible light and UV light, a color image that represents the portion of the environment illuminated by the emitted UV light and by visible light incident on the portion of the environment. The method may also include determining (i) a first extent to which UV light is attenuated in connection with pixels of the color camera that have a first color and (ii) a second extent to which UV light is attenuated in connection with pixels of the color camera that have a second color. The method may additionally include generating a UV image based on the color image, the first extent, and the second extent. The method may further include identifying one or more features of the environment by processing the UV image.

In a third example embodiment, a non-transitory computer-readable storage medium may have stored thereon instructions that, when executed by a computing device, cause the computing device to perform operations. The operations may include causing a UV illuminator to emit UV light towards a portion of an environment, and receiving, from a color camera configured to detect visible light and UV light, a color image that represents the portion of the environment illuminated by the emitted UV light and by visible light incident on the portion of the environment. The operations may also include determining (i) a first extent to which UV light is attenuated in connection with pixels of the color camera that have a first color and (ii) a second extent to which UV light is attenuated in connection with pixels of the color camera that have a second color. The operations may additionally include generating a UV image based on the color image, the first extent, and the second extent. The operations may further include identifying one or more features of the environment by processing the UV image.

In a fourth example embodiment, a system may include a means for generating color images that is configured to detect visible light and UV light, and a means for emitting UV light. The system may also include means for causing the means for emitting UV light to emit UV light towards a portion of an environment, and a means for receiving, from the means for generating color images, a color image that represents the portion of the environment illuminated by the emitted UV light and by visible light incident on the portion of the environment. The system may additionally include means for determining (i) a first extent to which UV light is attenuated in connection with pixels of the means for generating color images that have a first color and (ii) a second extent to which UV light is attenuated in connection with pixels of the means for generating color images that have a second color. The system further includes means for generating a UV image based on the color image, the first extent, and the second extent, and means for identifying one or more features of the environment by processing the UV image.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates a configuration of a robotic system, in accordance with example embodiments.

FIG. 2 illustrates a mobile robot, in accordance with example embodiments.

FIG. 3 illustrates an exploded view of a mobile robot, in accordance with example embodiments.

FIG. 4 illustrates a robotic arm, in accordance with example embodiments.

FIG. 5 illustrates UV imaging of an environment, in accordance with example embodiments.

FIG. 6A illustrates a system for generating UV images, in accordance with example embodiments.

FIG. 6B illustrates a system for generating UV images, in accordance with example embodiments.

FIG. 7 illustrates an optical system, in accordance with example embodiments.

FIG. 8 is a flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example,” “exemplary,” and/or “illustrative” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order. Unless otherwise noted, figures are not drawn to scale.

### I. OVERVIEW

An ultraviolet (UV) camera may be configured to generate UV images which may represent various features of an environment, some of which might not be visible within images captured using a different portion of the electromagnetic spectrum, such as visible light images and/or infrared (IR) images. However, the UV images generated by the UV camera do not represent the visible spectrum colors of the various features. This absence of color from UV images may make performance of some tasks based on UV images difficult. For example, some feature identification algorithms (e.g., feature detection, classification, segmentation, etc.) may rely on color to distinguish the feature from and/or contextualize the feature relative to other parts of the environment.

Thus, while UV images may allow for identification of some otherwise-invisible features, the absence of color may hinder identification of some features that might otherwise be represented in color in a visible light image. Accordingly, it may be desirable to capture a UV image of a portion of the environment that is aligned with a color (e.g., RGB) image of the same portion of the environment, such that there is at least a threshold amount of pixel-wise correspondence between the UV image and the color image. In particular, it is desirable for the UV image and the corresponding color image to be spatially aligned (i.e., both images representing approximately the same portion from approximately the same perspective) and temporally aligned (i.e., both images being captured at approximately the same time). Using this combination of images, both the UV contrast and the color of the various features of the environment may be represented and used in identifying and/or determining attributes of the various features.

One approach for capturing a UV image and a corresponding color image involves using a color camera, such as an RGB camera, to generate both the color image and the UV image of a scene, or portion of the environment. The color camera may include a color filter array (CFA), or may be configured to sense color in other ways, and the optical path thereof may be configured to allow for transmission of both UV light and visible light to the image sensor. Specifically, the color camera may include optical components (e.g., lenses, coatings, and/or filters) configured to (i) allow transmission of UV light and visible light and/or (ii) block transmission of other wavelengths outside of the UV and visible portions of the electromagnetic spectrum, thus allowing both visible light and UV light to reach the image sensor and be detected thereby without interference by other wavelengths.

In a first example, a UV illuminator may be configured to emit UV light towards the scene during a first time interval. While the scene is illuminated by the UV light, the color camera may be used to capture a first image of the scene. The color camera may also be used to capture a second image of the scene during a second time interval (e.g., preceding or subsequent to the first time interval) while the UV illuminator is not emitting UV light. In some cases, the UV illuminator may be caused to emit light with a power sufficiently high such that at least a threshold fraction (e.g., 50%) of the signal generated by the color camera may be substantially caused by the reflected UV light, while the rest is caused by the reflected visible light.

A UV image may be generated based on a difference image determined based on the first image and the second image. Specifically, the second image, which represents the scene under visible light illumination, may be subtracted from the first image, which represents the scene under UV and visible light illumination. In some cases, the difference image may constitute the UV image. In other cases, the difference image may be further processed to generate the UV image. For example, different colors of the color filter of the color camera and/or different pixels of the color camera may attenuate the UV light emitted by the UV illuminator to different extents (e.g., green pixels may be more transmissive of the UV light than blue pixels). The extent of attenuation may depend on the particular UV frequencies emitted by the UV illuminator.

Accordingly, in some implementations, the respective extent of attenuation of UV light associated with each pixel color (e.g., each of the red, green, and blue colored pixels) may be determined, and may be used to adjust different portions of the difference image accordingly. Specifically, different colors of the CFA may transmit the UV light to different extents, thus resulting in a different amount of UV light reaching a photosite of a given pixel depending on the color of the optical filter associated therewith. For example, the extent of attenuation associated with each color may be expressed in terms of a transparency of a given color to UV light, that is, as the signal generated by a pixel associated with the given color in response to to a portion of a scene that, absent the attenuation, would produce substantially a maximum (e.g., 250+/−5) pixel value. Accordingly, the adjustment of the difference image may involve dividing a corresponding value of each respective pixel of the difference image by the extent of attenuation associated with the color associated with the respective pixel.

In other implementations, the respective extent of attenuation may instead be determined on a pixel-by-pixel basis. Specifically, a calibration UV image representing a transparency to UV light of an optical path of each pixel may be generated by determining the response of each pixel to a corresponding portion of a scene that, absent the attenuation, would produce substantially the maximum signal value. In addition to quantifying the extent of attenuation due to the different colors of the CFA, determining the extent of attenuation in this way also allows for measurement of differences among pixels, in the response to UV light, caused by factors other than the presence of the CFA (e.g., manufacturing variations). Accordingly, the adjustment of the difference image may involve dividing a corresponding value of each respective pixel of the difference image by the value of a corresponding pixel of the calibration UV image.

In some cases, the environment may include features that exhibit fluorescence when illuminated by UV light. Thus, the second image may represent (i) the reflected UV light, (ii) reflected visible light, and (iii) visible light generated by the fluorescence. Accordingly, the difference image may represent (i) the reflected UV light and (iii) the visible light generated by the fluorescence. The UV image may be generated by demosaicing the difference image, resulting in the UV image also containing a color representation of the features that exhibit fluorescence.

In a second example, the UV image may be determined based on a single color image. Specifically, the color camera may be used to capture the (first) color image of the scene while the scene is illuminated by UV light emitted by the UV illuminator, as described above. Rather than relying on subtraction of a (second) color image from this (first) color image (which also represents reflection of the emitted UV light), the UV image may instead be determined computationally based on the (first) color image and the respective extents to which pixels of different colors attenuate the UV light.

In particular, a total signal of each pixel of the color image may include a visible light component and a UV light component. The visible light component and the UV light component may both be represented using the same one or more values corresponding to one or more color channels, and may thus be indistinguishable from one another. Additionally, the UV light component of each respective pixel may represent corresponding UV light that was incident on the color camera, transmitted through a corresponding optical path of the color camera, and detected by the photosite of the respective pixel. Thus, in order to determine a total UV light that, absent attenuation by the optical path of the respective pixel, would be detected by the respective pixel, the UV light component of the respective pixel may be adjusted according to the extent of attenuation associated with the color(s) represented by the respective pixel.

Accordingly, each pixel of the color image may be expressed as a product of a weighting matrix and one or more values of a corresponding pixel of a color-UV image. For example, the color image may be an W×H array of 3×1 matrices representing RGB values, the weighting matrix may be a 3×4 matrix based on the extents of UV light attenuation associated with different pixel colors, and the color-UV image may be an W×H array of 4×1 matrices each of which separately represents the RGB and UV values of a corresponding pixel of the color image. Since the weighting matrix used to transform the color-UV image to the color image is low-rank, the color-UV image may be determined, for example, by solving an optimization equation.

Specifically, the optimization equation may include a transformation loss function configured to determine a transformation loss value based on a difference between (i) the color image and (ii) the color-UV image as transformed by the weighting matrix. The optimization equation may also include a regularization function configured to generate a regularization loss value based on detection and/or quantification of the presence of one or more desired features and/or characteristics across one or more channels (e.g., R, G, B, and/or UV) of the color-UV image. In one example, the regularization function may measure a total variation of one of more channels of the color-UV image. In some implementations, the regularization function may be expressed as a machine learning model that has been trained to detect and/or quantify the one or more features and/or characteristics. The regularization function may thus provide an additional constraint for the optimization equation and thus facilitate determination of a satisfactory solution thereof.

The color-UV image may be iteratively adjusted until a total loss value of the optimization equation, which may be based on a sum of the transformation loss value and the regularization loss value, is reduced below a threshold loss value. The UV channel of the color-UV image may thus represent the UV image, while the color channels of the color-UV image may represent an adjusted color image that approximately represents ground-truth colors of the scene. In some implementations, the color-UV image may be generated prior to demosaicing of the color image, in which case each pixel of the color image may represent a single corresponding color value. Accordingly, the adjusted color image may be generated by demosaicing the color channels of the color-UV image. In other implementations, the color-UV image may be generated after demosaicing of the color image, in which case each pixel of the color image may represent one or more corresponding color values. Thus, the adjusted color image may be generated without demosaicing the color channels of the color-UV image.

Regardless of how the UV and/or color images are generated, the UV image and, in some cases, the color image may be processed to identify one or more features of the environment. For example, the one or more features may be parts of the environment that are to be cleaned due to being identified as unclean (e.g., constituting and/or containing visual patterns indicative of contaminants). In one example, the processing may be carried out by a machine learning model, such as an artificial neural network, that has been trained to detect the one or more features. In some cases, the machine learning model may be configured to process a depth-wise concatenation of the UV image and a corresponding color-only image (e.g., the adjusted color image and/or the second color image), thus concurrently considering representations of the features in the UV image and the color image.

### II. EXAMPLE ROBOTIC SYSTEMS

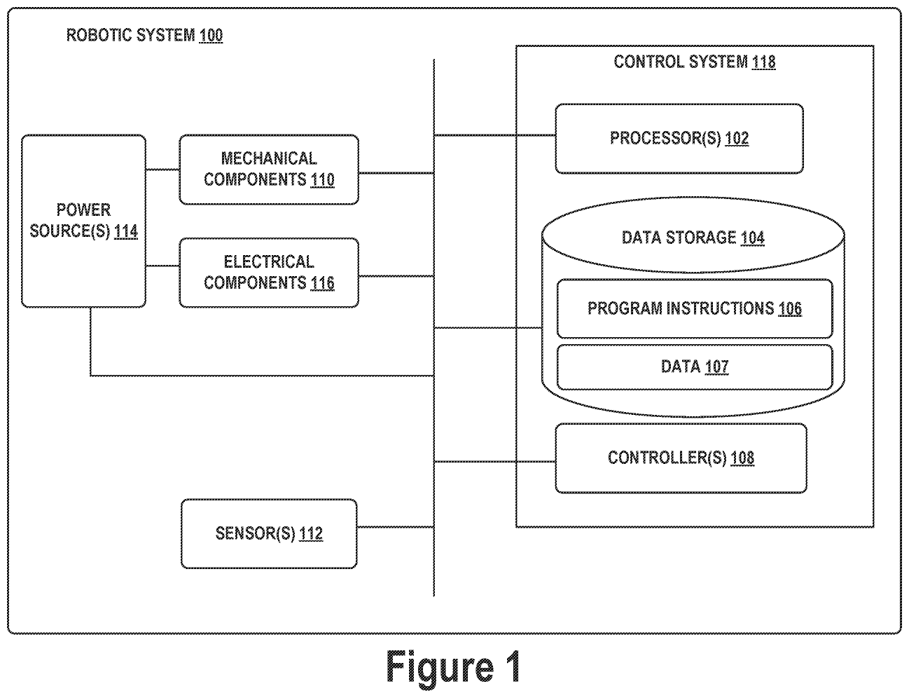

FIG. 1 illustrates an example configuration of a robotic system that may be used in connection with the implementations described herein. Robotic system 100 may be configured to operate autonomously, semi-autonomously, or using directions provided by user(s). Robotic system 100 may be implemented in various forms, such as a robotic arm, industrial robot, or some other arrangement. Some example implementations involve a robotic system 100 engineered to be low cost at scale and designed to support a variety of tasks. Robotic system 100 may be designed to be capable of operating around people. Robotic system 100 may also be optimized for machine learning. Throughout this description, robotic system 100 may also be referred to as a robot, robotic device, and/or mobile robot, among other designations.

As shown in FIG. 1, robotic system 100 may include processor(s) 102, data storage 104, and controller(s) 108, which together may be part of control system 118. Robotic system 100 may also include sensor(s) 112, power source(s) 114, mechanical components 110, and electrical components 116. Nonetheless, robotic system 100 is shown for illustrative purposes, and may include more or fewer components. The various components of robotic system 100 may be connected in any manner, including by way of wired or wireless connections. Further, in some examples, components of robotic system 100 may be distributed among multiple physical entities rather than a single physical entity. Other example illustrations of robotic system 100 may exist as well.

Processor(s) 102 may operate as one or more general-purpose hardware processors or special purpose hardware processors (e.g., digital signal processors, application specific integrated circuits, etc.). Processor(s) 102 may be configured to execute computer-readable program instructions 106 and manipulate data 107, both of which are stored in data storage 104. Processor(s) 102 may also directly or indirectly interact with other components of robotic system 100, such as sensor(s) 112, power source(s) 114, mechanical components 110, or electrical components 116.

Data storage 104 may be one or more types of hardware memory. For example, data storage 104 may include or take the form of one or more computer-readable storage media that can be read or accessed by processor(s) 102. The one or more computer-readable storage media can include volatile and/or non-volatile storage components, such as optical, magnetic, organic, or another type of memory or storage, which can be integrated in whole or in part with processor(s) 102. In some implementations, data storage 104 can be a single physical device. In other implementations, data storage 104 can be implemented using two or more physical devices, which may communicate with one another via wired or wireless communication. As noted previously, data storage 104 may include the computer-readable program instructions 106 and data 107. Data 107 may be any type of data, such as configuration data, sensor data, or diagnostic data, among other possibilities.

Controller 108 may include one or more electrical circuits, units of digital logic, computer chips, or microprocessors that are configured to (perhaps among other tasks), interface between any combination of mechanical components 110, sensor(s) 112, power source(s) 114, electrical components 116, control system 118, or a user of robotic system 100. In some implementations, controller 108 may be a purpose-built embedded device for performing specific operations with one or more subsystems of robotic system 100.

Control system 118 may monitor and physically change the operating conditions of robotic system 100. In doing so, control system 118 may serve as a link between portions of robotic system 100, such as between mechanical components 110 or electrical components 116. In some instances, control system 118 may serve as an interface between robotic system 100 and another computing device. Further, control system 118 may serve as an interface between robotic system 100 and a user. In some instances, control system 118 may include various components for communicating with robotic system 100, including a joystick, buttons, or ports, etc. The example interfaces and communications noted above may be implemented via a wired or wireless connection, or both. Control system 118 may perform other operations for robotic system 100 as well.

During operation, control system 118 may communicate with other systems of robotic system 100 via wired and/or wireless connections, and may further be configured to communicate with one or more users of the robot. As one possible illustration, control system 118 may receive an input (e.g., from a user or from another robot) indicating an instruction to perform a requested task, such as to pick up and move an object from one location to another location. Based on this input, control system 118 may perform operations to cause the robotic system 100 to make a sequence of movements to perform the requested task. As another illustration, a control system may receive an input indicating an instruction to move to a requested location. In response, control system 118 (perhaps with the assistance of other components or systems) may determine a direction and speed to move robotic system 100 through an environment en route to the requested location.

Operations of control system 118 may be carried out by processor(s) 102. Alternatively, these operations may be carried out by controller(s) 108, or a combination of processor(s) 102 and controller(s) 108. In some implementations, control system 118 may partially or wholly reside on a device other than robotic system 100, and therefore may at least in part control robotic system 100 remotely.

Mechanical components 110 represent hardware of robotic system 100 that may enable robotic system 100 to perform physical operations. As a few examples, robotic system 100 may include one or more physical members, such as an arm, an end effector, a head, a neck, a torso, a base, and wheels. The physical members or other parts of robotic system 100 may further include actuators arranged to move the physical members in relation to one another. Robotic system 100 may also include one or more structured bodies for housing control system 118 or other components, and may further include other types of mechanical components. The particular mechanical components 110 used in a given robot may vary based on the design of the robot, and may also be based on the operations or tasks the robot may be configured to perform.

In some examples, mechanical components 110 may include one or more removable components. Robotic system 100 may be configured to add or remove such removable components, which may involve assistance from a user or another robot. For example, robotic system 100 may be configured with removable end effectors or digits that can be replaced or changed as needed or desired. In some implementations, robotic system 100 may include one or more removable or replaceable battery units, control systems, power systems, bumpers, or sensors. Other types of removable components may be included within some implementations.

Robotic system 100 may include sensor(s) 112 arranged to sense aspects of robotic system 100. Sensor(s) 112 may include one or more force sensors, torque sensors, velocity sensors, acceleration sensors, position sensors, proximity sensors, motion sensors, location sensors, load sensors, temperature sensors, touch sensors, depth sensors, ultrasonic range sensors, infrared sensors, object sensors, or cameras, among other possibilities. Within some examples, robotic system 100 may be configured to receive sensor data from sensors that are physically separated from the robot (e.g., sensors that are positioned on other robots or located within the environment in which the robot is operating).

Sensor(s) 112 may provide sensor data to processor(s) 102 (perhaps by way of data 107) to allow for interaction of robotic system 100 with its environment, as well as monitoring of the operation of robotic system 100. The sensor data may be used in evaluation of various factors for activation, movement, and deactivation of mechanical components 110 and electrical components 116 by control system 118. For example, sensor(s) 112 may capture data corresponding to the terrain of the environment or location of nearby objects, which may assist with environment recognition and navigation.

In some examples, sensor(s) 112 may include RADAR (e.g., for long-range object detection, distance determination, or speed determination), LIDAR (e.g., for short-range object detection, distance determination, or speed determination), SONAR (e.g., for underwater object detection, distance determination, or speed determination), VICON® (e.g., for motion capture), one or more cameras (e.g., stereoscopic cameras for 3D vision), a global positioning system (GPS) transceiver, or other sensors for capturing information of the environment in which robotic system 100 is operating. Sensor(s) 112 may monitor the environment in real time, and detect obstacles, elements of the terrain, weather conditions, temperature, or other aspects of the environment. In another example, sensor(s) 112 may capture data corresponding to one or more characteristics of a target or identified object, such as a size, shape, profile, structure, or orientation of the object.

Further, robotic system 100 may include sensor(s) 112 configured to receive information indicative of the state of robotic system 100, including sensor(s) 112 that may monitor the state of the various components of robotic system 100. Sensor(s) 112 may measure activity of systems of robotic system 100 and receive information based on the operation of the various features of robotic system 100, such as the operation of an extendable arm, an end effector, or other mechanical or electrical features of robotic system 100. The data provided by sensor(s) 112 may enable control system 118 to determine errors in operation as well as monitor overall operation of components of robotic system 100.

As an example, robotic system 100 may use force/torque sensors to measure load on various components of robotic system 100. In some implementations, robotic system 100 may include one or more force/torque sensors on an arm or end effector to measure the load on the actuators that move one or more members of the arm or end effector. In some examples, the robotic system 100 may include a force/torque sensor at or near the wrist or end effector, but not at or near other joints of a robotic arm. In further examples, robotic system 100 may use one or more position sensors to sense the position of the actuators of the robotic system. For instance, such position sensors may sense states of extension, retraction, positioning, or rotation of the actuators on an arm or end effector.

As another example, sensor(s) 112 may include one or more velocity or acceleration sensors. For instance, sensor(s) 112 may include an inertial measurement unit (IMU). The IMU may sense velocity and acceleration in the world frame, with respect to the gravity vector. The velocity and acceleration sensed by the IMU may then be translated to that of robotic system 100 based on the location of the IMU in robotic system 100 and the kinematics of robotic system 100.

Robotic system 100 may include other types of sensors not explicitly discussed herein. Additionally or alternatively, the robotic system may use particular sensors for purposes not enumerated herein.

Robotic system 100 may also include one or more power source(s) 114 configured to supply power to various components of robotic system 100. Among other possible power systems, robotic system 100 may include a hydraulic system, electrical system, batteries, or other types of power systems. As an example illustration, robotic system 100 may include one or more batteries configured to provide charge to components of robotic system 100. Some of mechanical components 110 or electrical components 116 may each connect to a different power source, may be powered by the same power source, or be powered by multiple power sources.

Any type of power source may be used to power robotic system 100, such as electrical power or a gasoline engine. Additionally or alternatively, robotic system 100 may include a hydraulic system configured to provide power to mechanical components 110 using fluid power. Components of robotic system 100 may operate based on hydraulic fluid being transmitted throughout the hydraulic system to various hydraulic motors and hydraulic cylinders, for example. The hydraulic system may transfer hydraulic power by way of pressurized hydraulic fluid through tubes, flexible hoses, or other links between components of robotic system 100. Power source(s) 114 may charge using various types of charging, such as wired connections to an outside power source, wireless charging, combustion, or other examples.

Electrical components 116 may include various mechanisms capable of processing, transferring, or providing electrical charge or electric signals. Among possible examples, electrical components 116 may include electrical wires, circuitry, or wireless communication transmitters and receivers to enable operations of robotic system 100. Electrical components 116 may interwork with mechanical components 110 to enable robotic system 100 to perform various operations. Electrical components 116 may be configured to provide power from power source(s) 114 to the various mechanical components 110, for example. Further, robotic system 100 may include electric motors. Other examples of electrical components 116 may exist as well.

Robotic system 100 may include a body, which may connect to or house appendages and components of the robotic system. As such, the structure of the body may vary within examples and may further depend on particular operations that a given robot may have been designed to perform. For example, a robot developed to carry heavy loads may have a wide body that enables placement of the load. Similarly, a robot designed to operate in tight spaces may have a relatively tall, narrow body. Further, the body or the other components may be developed using various types of materials, such as metals or plastics. Within other examples, a robot may have a body with a different structure or made of various types of materials.

The body or the other components may include or carry sensor(s) 112. These sensors may be positioned in various locations on the robotic system 100, such as on a body, a head, a neck, a base, a torso, an arm, or an end effector, among other examples.

Robotic system 100 may be configured to carry a load, such as a type of cargo that is to be transported. In some examples, the load may be placed by the robotic system 100 into a bin or other container attached to the robotic system 100. The load may also represent external batteries or other types of power sources (e.g., solar panels) that the robotic system 100 may utilize. Carrying the load represents one example use for which the robotic system 100 may be configured, but the robotic system 100 may be configured to perform other operations as well.

As noted above, robotic system 100 may include various types of appendages, wheels, end effectors, gripping devices and so on. In some examples, robotic system 100 may include a mobile base with wheels, treads, or some other form of locomotion. Additionally, robotic system 100 may include a robotic arm or some other form of robotic manipulator. In the case of a mobile base, the base may be considered as one of mechanical components 110 and may include wheels, powered by one or more of actuators, which allow for mobility of a robotic arm in addition to the rest of the body.

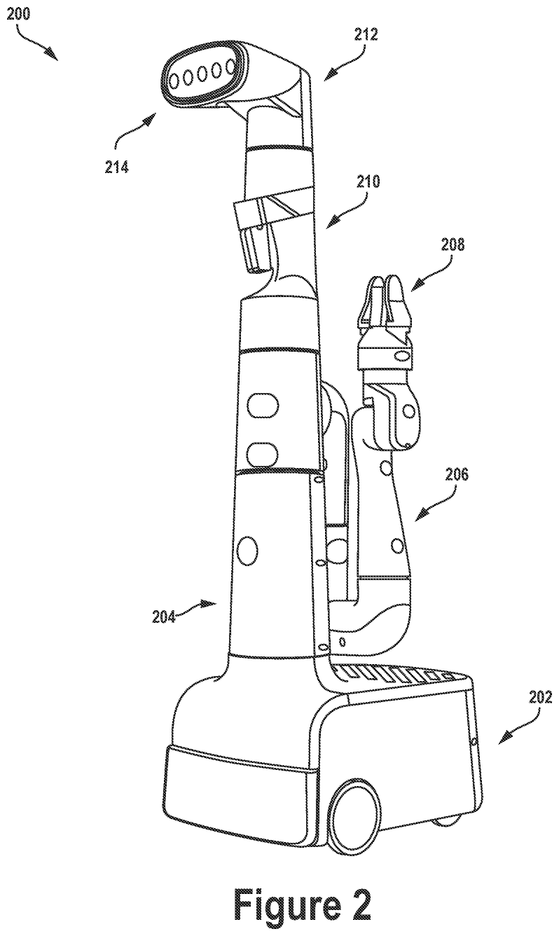

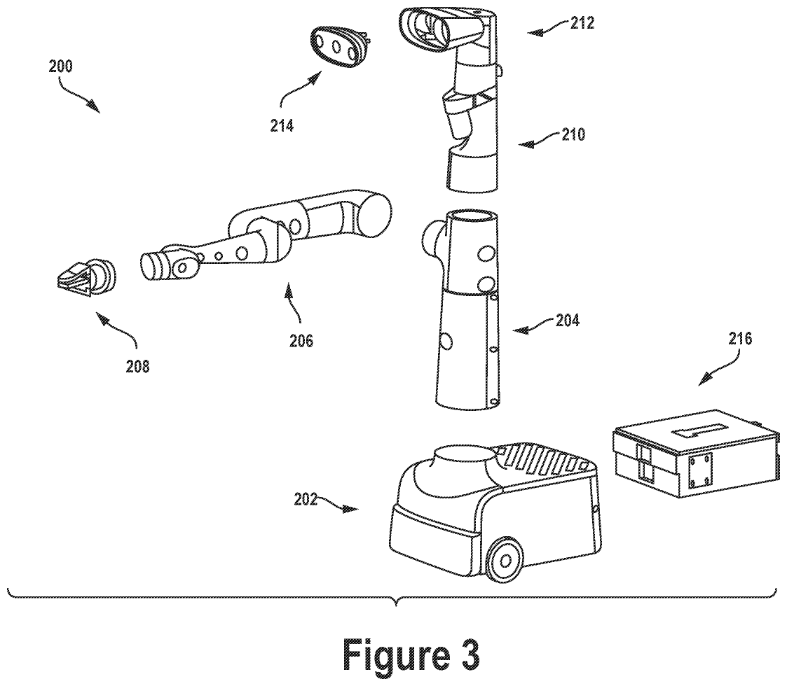

FIG. 2 illustrates a mobile robot, in accordance with example embodiments. FIG. 3 illustrates an exploded view of the mobile robot, in accordance with example embodiments. More specifically, robot 200 may include mobile base 202, midsection 204, arm 206, end-of-arm system (EOAS) 208, mast 210, perception housing 212, and perception suite 214. Robot 200 may also include compute box 216 stored within mobile base 202.

Mobile base 202 includes two drive wheels positioned at a front end of robot 200 in order to provide locomotion to robot 200. Mobile base 202 also includes additional casters (not shown) to facilitate motion of mobile base 202 over a ground surface. Mobile base 202 may have a modular architecture that allows compute box 216 to be easily removed. Compute box 216 may serve as a removable control system for robot 200 (rather than a mechanically integrated control system). After removing external shells, compute box 216 can be easily removed and/or replaced. Mobile base 202 may also be designed to allow for additional modularity. For example, mobile base 202 may also be designed so that a power system, a battery, and/or external bumpers can all be easily removed and/or replaced.

Midsection 204 may be attached to mobile base 202 at a front end of mobile base 202. Midsection 204 includes a mounting column which is fixed to mobile base 202. Midsection 204 additionally includes a rotational joint for arm 206. More specifically, Midsection 204 includes the first two degrees of freedom for arm 206 (a shoulder yaw J0 joint and a shoulder pitch J1 joint). The mounting column and the shoulder yaw J0 joint may form a portion of a stacked tower at the front of mobile base 202. The mounting column and the shoulder yaw J0 joint may be coaxial. The length of the mounting column of midsection 204 may be chosen to provide arm 206 with sufficient height to perform manipulation tasks at commonly encountered height levels (e.g., coffee table top and/or counter top levels). The length of the mounting column of midsection 204 may also allow the shoulder pitch J1 joint to rotate arm 206 over mobile base 202 without contacting mobile base 202.

Arm 206 may be a 7DOF robotic arm when connected to midsection 204. As noted, the first two DOFs of arm 206 may be included in midsection 204. The remaining five DOFs may be included in a standalone section of arm 206 as illustrated in FIGS. 2 and 3. Arm 206 may be made up of plastic monolithic link structures. Inside arm 206 may be housed standalone actuator modules, local motor drivers, and thru bore cabling.

EOAS 208 may be an end effector at the end of arm 206. EOAS 208 may allow robot 200 to manipulate objects in the environment. As shown in FIGS. 2 and 3, EOAS 208 may be a gripper, such as an underactuated pinch gripper. The gripper may include one or more contact sensors such as force/torque sensors and/or non-contact sensors such as one or more cameras to facilitate object detection and gripper control. EOAS 208 may also be a different type of gripper such as a suction gripper or a different type of tool such as a drill or a brush. EOAS 208 may also be swappable or include swappable components such as gripper digits.

Mast 210 may be a relatively long, narrow component between the shoulder yaw J0 joint for arm 206 and perception housing 212. Mast 210 may be part of the stacked tower at the front of mobile base 202. Mast 210 may be fixed relative to mobile base 202. Mast 210 may be coaxial with midsection 204. The length of mast 210 may facilitate perception by perception suite 214 of objects being manipulated by EOAS 208. Mast 210 may have a length such that when the shoulder pitch J1 joint is rotated vertical up, a topmost point of a bicep of arm 206 is approximately aligned with a top of mast 210. The length of mast 210 may then be sufficient to prevent a collision between perception housing 212 and arm 206 when the shoulder pitch J1 joint is rotated vertical up.

As shown in FIGS. 2 and 3, mast 210 may include a 3D LIDAR sensor configured to collect depth information about the environment. The 3D LIDAR sensor may be coupled to a carved-out portion of mast 210 and fixed at a downward angle. The LIDAR position may be optimized for localization, navigation, and for front cliff detection.

Perception housing 212 may include at least one sensor making up perception suite 214. Perception housing 212 may be connected to a pan/tilt control to allow for reorienting of perception housing 212 (e.g., to view objects being manipulated by EOAS 208). Perception housing 212 may be a part of the stacked tower fixed to mobile base 202. A rear portion of perception housing 212 may be coaxial with mast 210.

Perception suite 214 may include a suite of sensors configured to collect sensor data representative of the environment of robot 200. Perception suite 214 may include an infrared (IR)-assisted stereo depth sensor. Perception suite 214 may additionally include a wide-angled RGB camera for human-robot interaction and context information. Perception suite 214 may additionally include a high resolution RGB camera for object classification. A face light ring surrounding perception suite 214 may also be included for improved human-robot interaction and scene illumination. In some examples, perception suite 214 may also include a projector configured to project images and/or video into the environment.

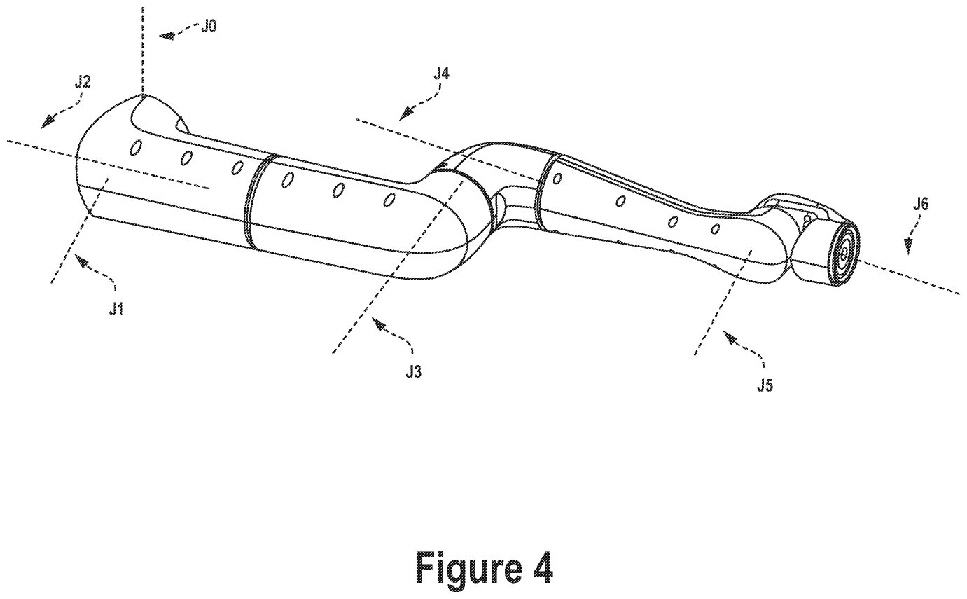

FIG. 4 illustrates a robotic arm, in accordance with example embodiments. The robotic arm includes 7 DOFs: a shoulder yaw J0 joint, a shoulder pitch J1 joint, a bicep roll J2 joint, an elbow pitch J3 joint, a forearm roll J4 joint, a wrist pitch J5 joint, and wrist roll J6 joint. Each of the joints may be coupled to one or more actuators. The actuators coupled to the joints may be operable to cause movement of links down the kinematic chain (as well as any end effector attached to the robot arm).

The shoulder yaw J0 joint allows the robot arm to rotate toward the front and toward the back of the robot. One beneficial use of this motion is to allow the robot to pick up an object in front of the robot and quickly place the object on the rear section of the robot (as well as the reverse motion). Another beneficial use of this motion is to quickly move the robot arm from a stowed configuration behind the robot to an active position in front of the robot (as well as the reverse motion).

The shoulder pitch J1 joint allows the robot to lift the robot arm (e.g., so that the bicep is up to perception suite level on the robot) and to lower the robot arm (e.g., so that the bicep is just above the mobile base). This motion is beneficial to allow the robot to efficiently perform manipulation operations (e.g., top grasps and side grasps) at different target height levels in the environment. For instance, the shoulder pitch J1 joint may be rotated to a vertical up position to allow the robot to easily manipulate objects on a table in the environment. The shoulder pitch J1 joint may be rotated to a vertical down position to allow the robot to easily manipulate objects on a ground surface in the environment.

The bicep roll J2 joint allows the robot to rotate the bicep to move the elbow and forearm relative to the bicep. This motion may be particularly beneficial for facilitating a clear view of the EOAS by the robot's perception suite. By rotating the bicep roll J2 joint, the robot may kick out the elbow and forearm to improve line of sight to an object held in a gripper of the robot.

Moving down the kinematic chain, alternating pitch and roll joints (a shoulder pitch J1 joint, a bicep roll J2 joint, an elbow pitch J3 joint, a forearm roll J4 joint, a wrist pitch J5 joint, and wrist roll J6 joint) are provided to improve the manipulability of the robotic arm. The axes of the wrist pitch J5 joint, the wrist roll J6 joint, and the forearm roll J4 joint are intersecting for reduced arm motion to reorient objects. The wrist roll J6 point is provided instead of two pitch joints in the wrist in order to improve object rotation.

### III. EXAMPLE UV IMAGING PROCESS

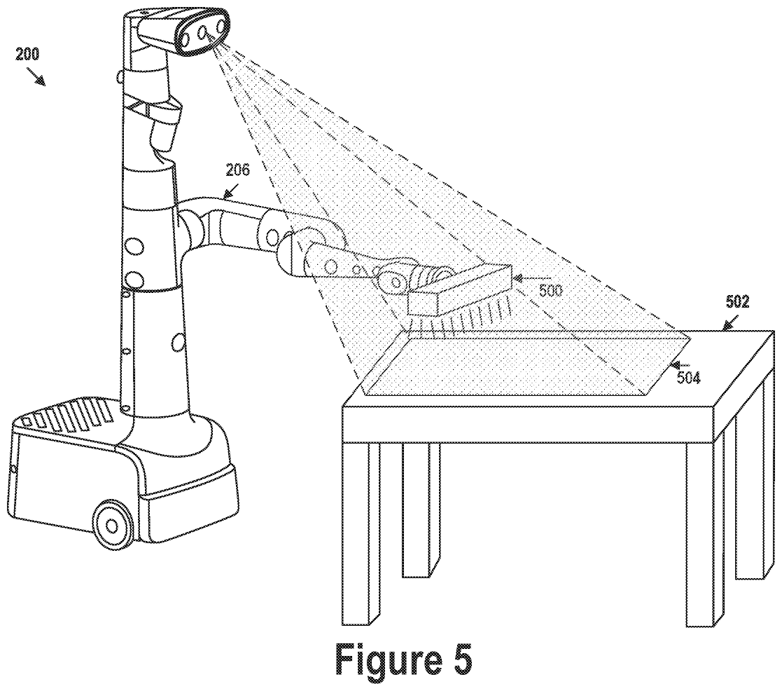

FIG. 5 illustrates an example scenario in which a UV illuminator is used in connection with capturing of a UV image, which may be processed to identify a feature (e.g., dirt, residue, foreign substance, or another material) present in an environment. Specifically, FIG. 5 shows robot 200 operating in an environment that contains therein table 502. Robot 200 includes UV illuminator 500 connected to an end of arm 206. Thus, in the arrangement shown, EOAS 208 may include UV illuminator 500. Robot 200 is shown scanning a portion of table 502, as indicated by field of view 504, while UV illuminator 500 is used to emit UV light towards at least part of table 502, as indicated by the lines projected from the bottom of UV illuminator 500. The UV light emitted by UV illuminator 500 may include UV-C light having wavelengths from 100 nanometers to 280 nanometers, UV-B light having wavelengths from 281 nanometers to 320 nanometers, and/or UV-A light having wavelengths from 321 nanometers to 400 nanometers.

Field of view 504 may correspond to a camera provided as part of perception suite 214 and configured to sense at least UV light. In some implementations, the camera may be disposed at a different location on robot 200, such as at the end of arm 206. Thus, in some cases, the camera and UV illuminator 500 may be co-located, may be simultaneously repositionable by way of arm 206, and/or may have the same or similar fields of view.

In some implementations, the camera associated with field of view 504 may be a color camera configured to generate images that represent UV light and visible light. Components provided along an optical path of the color camera may be configured to transmit UV light and visible light. That is, the color camera might lack components configured to block UV light and/or visible light (e.g., UV-blocking coatings on lenses). For example, the color camera may be a red-green-blue (RGB) camera that is configured to distinguish colors using a color filter array (CFA), such as a Bayer filter, or using an image sensor having vertically stacked photosites with varying spectral sensitivity, such as the FOVEON X3®. In some implementations, the camera may include components that are adapted for transmitting and/or sensing particular UV light wavelength(s) (e.g., wavelengths between 320 and 400 nanometers). For example, the camera may include one or more lenses, which may be made out of fused silica, fused quartz, and/or calcium fluoride, and thus configured to transmit UV light. The camera may also include optical filters configured to transmit the UV light and visible light and block at least some other type of light (e.g., infrared light).

When imaged using the visible portion of the electromagnetic spectrum (e.g., wavelengths between 400 and 740 nanometers), the tabletop of table 502 may appear clean, as shown in FIG. 5. Imaging the tabletop of table 502 using UV light, however, may reveal the presence of various stains on the tabletop of table 502. Specifically, some substances/materials may absorb or reflect UV light to a different extent than they absorb or reflect visible light, resulting in such substances/materials having a different appearance under UV illumination than under visible light illumination. For example, some substances/materials that would not appear in visible light images may appear in UV images (e.g., appear dark in UV images due to the substances/materials absorbing UV light, or appear bright in UV images due to the substances/materials reflecting UV light). Similarly, some aspects of a scene might be visible under visible light illumination, but might not be visible under UV illumination. Accordingly, it may be desirable to capture a UV image that is aligned with (i.e., pixel-wise corresponds to) a corresponding color image, since such a pair of images may allow for detection of a wider range of possible features in a scene.

In some implementations, rather than using a dedicated UV image sensor to generate UV images, UV images may instead be generated by way of a visible light (color) image sensor, such as an RGB camera. In one example, the power with which UV illuminator 500 emits the UV light may be increased to a point where the amount of UV light captured by the visible light image sensor sufficiently exceeds the amount of visible light captured thereby, resulting in the corresponding image data representing more UV light than visible light (e.g., (UV light)/(visible light)>1.0). Accordingly, when more than a threshold extent of the signal represented by the color image is caused by the emitted UV light, the color image may, without additional processing, be considered to be a UV image.

In another example, the visible light image sensor may (while the camera and scene remain stationary) be used to capture two images: one image with UV illumination and one image without UV illumination. The image without UV illumination may be subtracted from the image with UV illumination, resulting in a difference image that represents primarily the reflected UV light. In a further example, each color of a Bayer filter of the visible light image sensor may block UV light to a different extent. Thus, the signal generated by each pixel of the visible light image sensor may be weighted according to the color of its corresponding Bayer filter region. Specifically, when demosaicing the UV image from pixel data, pixels associated with color filters that are configured to transmit relatively more UV light (e.g., filters configured to transmit red light) may be weighted more heavily than pixels associated with color filters that are configured to transmit relatively less UV light (e.g., filters configured to transmit green or blue light).

### IV. EXAMPLE SYSTEMS FOR GENERATING UV IMAGES

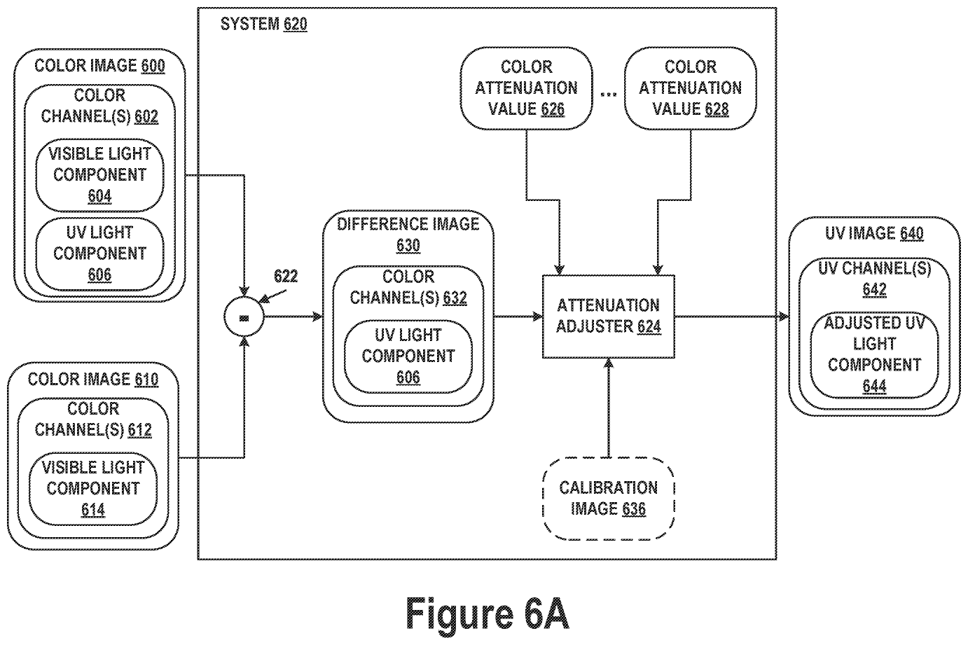

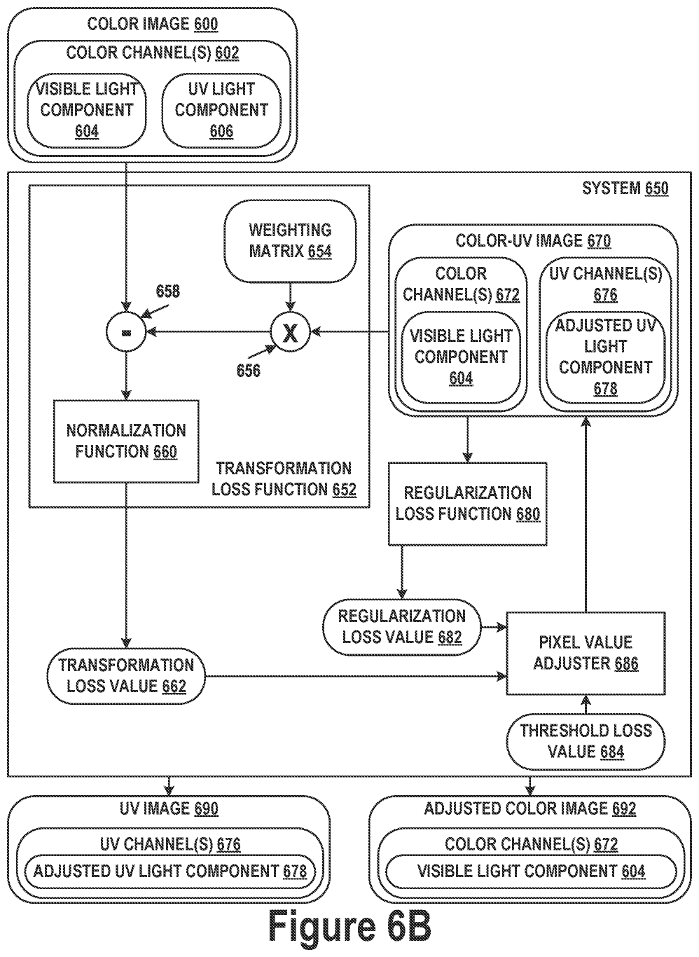

FIG. 6A illustrates a system for generating a UV image based on image data captured using a color camera. Specifically, system 620 may include difference operator 622, and attenuation adjuster 624, and may be configured to generate UV image 640 based on color image 600 and color image 610. Color images 600 and 610 may be, for example, RGB images generated by an RGB camera. Accordingly, color channel(s) 602 and 612 of color images 600 and 610, respectively, may each include a red channel configured to represent red values of pixels of the respective image, a green channel configured to represent green values of pixels of the respective image, and a blue channel configured to represent blue values of pixels of the respective image.

Color image 600 may be captured while the scene represented thereby is illuminated with UV light emitted by a UV illuminator (e.g., UV illuminator 500). Accordingly, color channel(s) 602 may be used to represent, for each respective pixel of color image 600, visible light component 604 of a corresponding signal generated by the respective pixel and UV light component 606 of the corresponding signal. Without further processing of the values of color channel(s) 602, visible light component 604 might not be distinguishable from UV light component 606, since both components may be represented using the same set of values of color channel(s) 602.

Color image 610 may be captured while the scene represented thereby is not illuminated with UV light emitted by the UV illuminator. Accordingly, color channel(s) 612 may be used to represent, for each respective pixel of color image 610, visible light component 614 of a corresponding signal generated by the respective pixel. Accordingly, color image 610 may be referred to as a color-only image in that it does not contain a UV light component caused by the UV illuminator. While both color images 600 and 610 may also represent the scene illuminated by environmental UV light (e.g., due to solar radiation), the effect of this environmental UV light is ignored for the purpose of clarity of this description, and because a power of the UV light emitted by the UV illuminator may sufficiently exceed a power of the environmental UV light so as to render negligible the contribution of the environmental UV light.

In some implementations, a capture time of color image 600 might differ from a capture time of color image 610 by no more than a threshold time period (e.g., 4 milliseconds, 9 milliseconds, 17 milliseconds, 34 milliseconds), such that color images 600 and 610 represent the scene at substantially the same time, and may thus be considered temporally aligned. For example, color images 600 and 610 may be captured sequentially, one after the other without any intervening images therebetween, and might thus be separated by a time equal to an inverse of the frame rate of the color camera. Additionally, color images 600 and 610 may be captured without repositioning the color camera (e.g., directly, or by moving a robotic device to which the color camera is connected) in between capturing of these images to represent substantially the same scene using both images. Although some features of the scene (e.g., mobile actors) may move between capturing of color image 600 and 610, such movement might not exceed, for example, a threshold distance in pixel space at least for features located within a threshold distance of the color camera. Such spatial and temporal alignment may result in at least a threshold extent of pixel-wise correspondence, and thus facilitate accurate color image subtraction by system 620.

Specifically, difference operator 622 of system 620 may be configured to subtract color image 610 from color image 600, thereby generating difference image 630. For example, difference operator 622 may be configured to subtract, for each respective pixel of color image 600, a value of a corresponding pixel in color image 610 from a value of the respective pixel in color image 600. When color images 600 and 610 are spatially and temporally aligned (i.e., exhibit pixel-wise correspondence), visible light component 604 may be equal (e.g., exactly or substantially) to visible light component 614. Thus, color channel(s) 632 of difference image 630 may represent UV light component 606, and might not represent visible light components 604 and/or 614. For example, difference image 630 may represent substantially only UV light component 606 in that more than a threshold portion (e.g., 90%, 95%, 99%, etc.) of the signal of color channel(s) 632 may represent UV light component 606, while a remaining portion represents aspects of visible light components 604 and/or 614.

Difference image 630 may be provided as input to attenuation adjuster 624, which may be configured to generate, based on difference image 630, UV image 640. Attenuation adjuster 624 may be configured to adjust values of color channel(s) 632 based on respective extents to which UV light is attenuated in connection with different pixels of the color camera. UV image 640 may include UV channel(s) 642, which may represent adjusted UV light component 644. Adjusted UV light component 644 may represent UV light component 606 adjusted based on the extents to which UV light is attenuated in connection with different pixels of the color camera, and may thus be a more accurate representation of the UV light reflected from the scene. In some implementations, attenuation adjuster 624 or another component (not shown) of system 620 may be configured to merge multiple color channels of color channel(s) 632 into a single UV channel 642 (e.g., since the notion of UV “color” might not be useful or meaningful).

In one example, attenuation adjuster 624 may be configured to adjust the values of color channel(s) 632 based on the corresponding color associated with each value. Specifically, the structures that allow different colors of visible light to be differentiated from one another may also attenuate the UV light to different extents. For example, for a particular range of UV wavelengths, red pixels of an RGB camera may attenuate the UV light to a lesser extent than green pixels of the RGB camera.

The extent of attenuation experienced in connection with pixels of a first color (e.g., red) may be represented by color attenuation value 626, and the extent of attenuation experienced in connection with pixels of a second color (e.g., blue) may be represented by color attenuation value 628, with the extent of attenuation experienced in connection with pixels of other colors possibly being represented by other attenuation values, as indicated by the ellipsis. Color attenuation values 626 through color attenuation value 628 (i.e., color attenuation values 626-628) may collectively represent the extent of attenuation for each different pixel color of the color camera (e.g., red, green, and blue). In some implementations, each of color attenuation values 626 -628 may include a plurality of attenuation values, with each attenuation value being specific to a particular UV wavelength and/or range of UV wavelengths. Accordingly, attenuation adjuster 624 may be configured to select a subset of the plurality of attenuation values depending on the specific wavelength(s) of UV light emitted by the UV illuminator.

In some implementations, color attenuation values 626-628 may represent, for each respective color, a fraction of UV light collected by the color camera that is absorbed by components of the color camera prior to detection of the collected UV light by pixels of the color camera. Thus, color attenuation values 626-628 may be considered to represent an opacity of components of the color camera to UV light. In other implementations, color attenuation values 626-628 may instead represent, for each respective color, a fraction of UV light collected by the color camera that is not absorbed by components of the color camera, and thus detected by the pixels of the color camera. Thus, color attenuation values 626-628 may be considered to represent a transparency of components of the color camera to UV light. Since transparency and opacity are complements, specification of one may also be indicative of the other.

For example, a transparency of red, green, and blue pixels to UV light may be represented as T_{RED}=α, T_{GREEN}=β, and T_{BLUE}=γ, respectively, corresponding to color attenuation values 626-628. Accordingly, Rᵢ^{ADJUSTED}=Rᵢ^{MEASURED}/α, Gᵢ^{ADJUSTED}=Gᵢ^{MEASURED}/β, and Bᵢ^{ADJUSTED}=Bᵢ^{MEASURED}/γ, where R represents a red-colored pixel, G represents a green-colored pixel, and B represents a blue-colored pixel, the subscript i indicates the ith pixel of a given color in the color image and iterates through all pixels of the given color, the superscript MEASURED indicates the value as measured by the image sensor and represented as part of difference image 630, and the superscript ADJUSTED indicates the value as determined by attenuation adjuster 624 based on the corresponding color-specific extent of UV light attenuation. An opacity of red, green, and blue pixels to UV light may be represented as O_{RED}=(1−α), O_{GREEN}=(1−β), and O_{BLUE}=(1−γ), respectively.

In another example, attenuation adjuster 624 may be configured to adjust the values of color channel(s) 632 in a pixel-specific, rather than a color-specific manner. Specifically, in addition to color-specific differences in UV light attenuation among pixels, UV light may additionally be attenuated to varying extents in connection with different pixels as a result of, for example, manufacturing variations in optical components of the camera and/or component degradation over time, among other factors. Accordingly, rather than relying on color attenuation values 626-628 to adjust difference image 630, attenuation adjuster 624 may instead be configured to adjust pixel values of difference image 630 based on calibration image 636.

Calibration image 636 may include the same number and arrangement of pixels as difference image 630. Thus, for each respective pixel of difference image 630, calibration image 636 may include a corresponding pixel indicating an extent to which UV light is attenuated in connection with the respective pixel. Calibration image 636 may be generated empirically by capturing, using the color camera, an image of a scene that, absent the attenuation of UV light by components of the color camera, would result in each pixel generating substantially a maximum value (e.g., a value of 250+/−5, where the pixel value ranges from 0 to 255). The scene may be configured such that substantially the maximum pixel value is achieved without saturating the pixel. Whether the scene is expected to produce a maximum pixel value may be evaluated using a UV image sensor prior to determining calibration image 636 using the color camera.

The color camera may capture calibration image 636 using the same or similar settings as used during capture of color images 600 and 610. Calibration image 636 may additionally or alternatively be generated by determining, for each pixel thereof, a UV light irradiance, for a given exposure time, associated with generating a maximum pixel value in the presence of attenuation, and/or an exposure time, at a given irradiance level, associated with generating the maximum pixel value, among other possibilities.

As with color attenuation values 626-628, each value of calibration image 636 may be expressed as an opacity or as a transparency. For example, a transparency of pixel cᵢ to UV light, where cᵢ∈C and C represents calibration image 636, may be represented as Tᵢ=δᵢ, while an opacity of pixel cᵢ to UV light may be represented as Oᵢ=1−δᵢ. Accordingly, pᵢ^{ADJUSTED}=pᵢ^{MEASURED}/δᵢ, where pᵢ^{MEASURED} represents an ith pixel of difference image 630 and pᵢ^{ADJUSTED} represents an ith pixel of UV image 640. Accordingly, when each pixel of calibration image 636 represents a transparency thereof to UV light, UV image 640 may be expressed as V=D/C, where V represents UV image 640, D represents difference image 630, and C represents calibration image 636.

FIG. 6B illustrates another system for generating a UV image based on image data captured using a color camera. Specifically, system 650 includes transformation loss function 652, regularization loss function 680, and pixel value adjuster 686. System 650 may be configured to generate UV image 690 and adjusted color image 692 based on color image 600. Unlike system 620, system 650 might not rely on a second color image (e.g., color image 610) in order to generate UV image 690. Instead, system 650 may be configured to computationally decompose each of the values of color channel(s) 602 into at least two values that represent visible light component 604 separately from UV light component 606.

For each respective pixel of color image 600, a value of a red color channel of the respective pixel may be expressed as Rᵢ^{MEASURED}=Rᵢ^{CORRECTED}+αUᵢ, where the subscript i indicates the respective pixel, the superscript MEASURED indicates the value actually measured by the photosite of the respective pixel, the superscript CORRECTED indicates an amount of visible light that was actually incident on the respective pixel (e.g., the red light component of Rᵢ^{MEASURED}), α indicates a transparency of the optical paths of red pixels of the color camera to UV light (as discussed above), and Uᵢ indicates an amount of UV light that was actually incident on the respective pixel (e.g., the adjusted UV component of Rᵢ^{MEASURED}). Similarly, for each respective pixel of color image 600, a value of a green color channel of the respective pixel may be expressed as Gᵢ^{MEASURED}=Gᵢ^{CORRECTED}+βUᵢ, and a value of a blue color channel of the respective pixel may be expressed as Bᵢ^{MEASURED}=Bᵢ^{CORRECTED}+γUᵢ, where β and γ indicate a transparency of the optical path of green pixels and blue pixels, respectively, of the color camera to UV light (as discussed above).

Accordingly, each pixel of color image 600 may be expressed as bᵢ=

$$[ \begin{matrix}{R}_{i}^{MEASURED} \\ {G}_{i}^{MEASURED} \\ {B}_{i}^{MEASURED}\end{matrix} ] = [ \begin{matrix}1 & 0 & 0 & α \\ 0 & 1 & 0 & β \\ 0 & 0 & 1 & γ\end{matrix} ] [ \begin{matrix}{R}_{i}^{CORRECTED} \\ {G}_{i}^{CORRECTED} \\ {B}_{i}^{CORRECTED} \\ {U}_{i}\end{matrix} ] ,$$

where

$$A = [ \begin{matrix}1 & 0 & 0 & α \\ 0 & 1 & 0 & β \\ 0 & 0 & 1 & γ\end{matrix} ]$$

represents weighting matrix 654, and

$${x}_{i} = [ \begin{matrix}{R}_{i}^{CORRECTED} \\ {G}_{i}^{CORRECTED} \\ {B}_{i}^{CORRECTED} \\ {U}_{i}\end{matrix} ]$$

represents an ith pixel of color-UV image 670. Specifically, Rᵢ^{CORRECTED}, Gᵢ^{CORRECTED}, and Bᵢ^{CORRECTED} may correspond to color channel(s) 672, and thus represent visible light component 604 of the ith pixel, while Uᵢ may correspond to UV channel(s) 676 and thus represent adjusted UV light component 678 of the ith pixel. Since bᵢ (pixel values of color image 600) and A (weighting matrix 654) are known, and xᵢ (pixel values of color-UV image 670) is unknown, xᵢ may be determined by solving the equation Axᵢ−bᵢ=0.

However, since A is low-rank, the equation Axᵢ−bᵢ=0 might not have a unique solution, and a satisfactory solution may instead be determined by solving an optimization problem. Solving the optimization problem may involve iteratively determining xᵢ for each pixel of color-UV image 670 until a total loss value of one or more loss functions is reduced to below a threshold value. Specifically, the value xᵢ of each respective pixel of color-UV image 670 may be determined by pixel value adjuster 686 based on threshold loss value 684, transformation loss value 662, and regularization loss value 682.

Specifically, transformation loss function 652 may be configured to determine transformation loss value 662, which may be expressed as Σₓ_{ᵢ}_{∈X}∥Axᵢ−bᵢ∥₂²=∥AX−B∥₂², where X represents color-UV image 670, B represents color image 600, xᵢ∈X, and bᵢ∈B. Thus, transformation loss function 652 may be configured to determine, for each pixel of color-UV image 670, a product of weighting matrix 654 and the respective pixel, as indicated by matrix multiplication operator 656. Transformation loss function 652 may also be configured to determine, for each respective pixel, a difference (e.g., using the L-2 norm or another norm) between the product and a corresponding pixel of color image 600, as indicated by difference operator 658. Transformation loss function 652 may additionally be configured to determine, for each respective pixel, normalization of the difference determined by difference operator 658, as indicated by normalization function 660. Normalization function 660 may include one or more additional operations applied to the output of difference operator 658. In the example provided above, normalization function 660, represented by the operator ∥⋅∥², indicates the difference raised to the power of two, resulting in a positive range of outputs. Accordingly, transformation loss function 652 may quantify an amount of signal, or visual information, lost by color-UV image 670 as transformed by weighting matrix 654 relative to color image 600.

Since the optimization problem discussed above may include a plurality of possible solutions, system 650 may also include regularization loss function 680, which may be configured to facilitate selection of at least one solution from the plurality of possible solutions. Specifically, regularization loss function 680 may be configured to determine regularization loss value 682 based on color-UV image 670, and may be represented as f_{REG}(X). Regularization loss function 680 may be configured to quantify the extent to which one or more visual features, visual patterns, and/or visual characteristics are present and/or absent across one or more channels of an image, such as color-UV image 670.

In one example, regularization loss function 680 may include a total variation denoising function (e.g., based on the Rudin, Osher, and Fatemi model), which may be configured to quantify an absolute gradient of one or more channels of color channel(s) 672 and/or UV channel(s) 676. In another example, regularization loss function 680 may include one or more filters configured to detect, for example, edges, blobs, or other visual features across two or more channels of color-UV image 670, and a comparator configured to quantify an extent to which the edges, blobs, or other visual features are aligned across the two or more channels. In a further example, regularization loss function 680 may include a machine learning model that has been trained to generate regularization loss value 682 based on processing of color-UV image 670. For example, the machine learning model may have been trained to quantify the extent to which the one or more visual features, visual patterns, and/or visual characteristics are present and/or absent across one or more channels of color-UV image 670.

Pixel value adjuster may be configured to determine a total loss value based on a weighted sum of transformation loss value 662 and regularization loss value 682. For example, the total loss value may be expressed as L_{TOTAL}=L_{TRANSFORMATION}+λL_{REGULARIZATION}, where the parameter λ is used to control a relative importance between transformation loss value 662 and regularization loss value 682. When the total loss value L_{TOTAL} exceeds threshold loss value 684, pixel value adjuster 686 may be configured to adjust one or more pixel values of color-UV image 670, thereby generating a new/updated version of color-UV image 670, in an attempt to reduce the the total loss value L_{TOTAL} to below threshold loss value 684.

Generation of new/updated versions of color-UV image 670 may be repeated by pixel value adjuster 686 until the total loss value L_{TOTAL} falls below threshold loss value 684, which may indicate that color-UV image 670 approximates color image 600 with no more than a threshold information loss, as quantified by loss functions 652 and 680. Pixel value adjuster 686 may be configured to adjust the one or more pixel values of color-UV image 670 using gradient descent, simulated annealing, genetic algorithms, and/or particle swarm optimization, among other possible approaches for solving optimization problems.

Based on and/or in response to generating a version of color-UV image 670 that produces a total loss value L_{TOTAL} that is below threshold loss value 684, system 650 may be configured to generate UV image 690 and adjusted color image 692. Specifically, the total loss value L_{TOTAL} being below threshold loss value 684 may indicate that the corresponding version of color-UV image 670 represents an accurate decomposition of color image 600 into distinct and separate color and UV values, as represented by color channel(s) 672 and UV channel(s) 676. Accordingly, UV image 690 may include UV channel(s) 676 representing adjusted UV light component 678, while adjusted color image 692 may include color channel(s) 672 representing visible light component 604.

Additionally or alternatively, color-UV image 670 may instead be determined by solving the equation Axᵢ−bᵢ=0 using other techniques. For example, this equation may be rewritten as xᵢ=(A⁺)bᵢ and solved directly using the Moore-Penrose inverse, where A⁺ represents a pseudoinverse of A. In some implementations, the Moore-Penrose inverse may be used in combination with the optimization techniques discussed above. For example, the Moore-Penrose inverse may be used to initialize the values of weighting matrix A, and these values may subsequently be refined using the optimization techniques discussed above.

Systems 620 and 650 may operate on color image 600 (and color image 610 in the case of system 620) either before or after demosaicing of color image 600. Operating on color image 600 prior to demosaicing may reduce a number of operations involved in generating the respective UV images relative to operating on color image 600 after demosaicing since, prior to demosaicing, each pixel of color image 600 represents one color rather than up to three colors. Accordingly, in implementations where the color camera outputs a demosaiced image, systems 620 and/or 650 may be configured to reverse the demosaicing operation to generate a mosaiced color image 600. Additionally, in some implementations, UV image 640, UV image 690, and/or adjusted color image 692 may be demosaiced by the respective systems prior to being output thereby.

### V. OPTICAL SYSTEM FOR GENERATING UV IMAGES

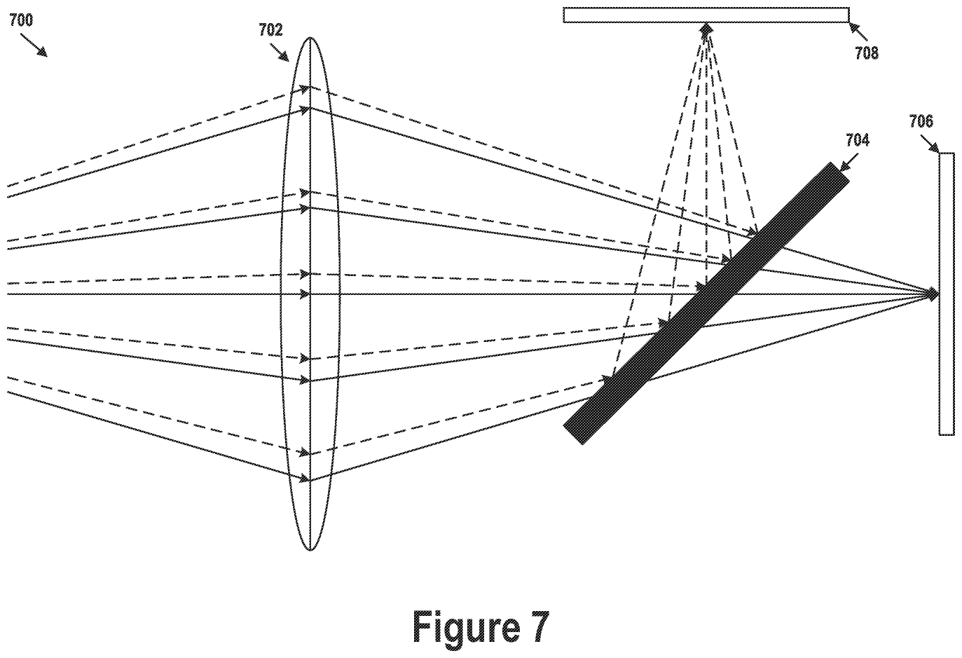

FIG. 7 illustrates an optical system for generating UV and color images using separate image sensors. Specifically, FIG. 7 illustrates optical system 700 that includes objective lens 702, wavelength splitter 704, color image sensor 706, and UV image sensor 708. Optical system 700 may define and/or form part of an image capture apparatus. Objective lens 702 may be configured to receive from the environment visible light, as represented by solid lines, and UV light, as represented by dashed lines. Wavelength splitter 704 may be positioned relative to objective lens 702 to receive therefrom both the visible and UV light collected thereby. In the example arrangement of FIG. 7, the visible and UV light from the environment may be incident of a left (first) side of objective lens 702, and may be directed to wavelength splitter 704 positioned on a right (second) side of objective lens 702.

Wavelength splitter 704 may be configured to pass the visible light and reflect the UV light. For example, wavelength splitter 704 may include a glass substrate coated with one or more optical filter materials configured to pass visible light and reflect UV light for a corresponding range of light incidence angles. Additionally, wavelength splitter 704 may be configured to direct the UV light along a first optical path onto UV image sensor 708 and the visible light along a second optical path onto color image sensor 706. For example, wavelength splitter 704 may be rotated (e.g., 45-degrees) relative to an optical axis of objective lens 702, resulting in the reflected UV light being focused behind objective lens 702, rather than being reflected back out of objective lens 702. In some cases, the angle between wavelength splitter 704 and the optical axis may be selected to achieve a range of light incidence angles that result in reflection of UV light and transmission of visible light.

In some implementations, optical system 700 may include additional components (not shown in FIG. 7) to facilitate the capture of UV and color images by image sensors 708 and 706, respectively. For example, a first additional lens may be positioned along the first optical path between UV image sensor 708 and wavelength splitter 704, and a second additional lens may be positioned along the second optical path between color image sensor 706 and wavelength splitter 704.

Since both the visible light and the UV light incident on image sensors 706 and 708, respectively, are initially captured using objective lens 702, both image sensors 706 and 708 may share the same field of view of the environment. Accordingly, UV images generated by UV image sensor 708 may be aligned with and/or may have a predetermined offset relative to color images generated using color image sensor 706. In some cases, image sensors 706 and 708 may share the same resolution. Additionally, image sensors 706 and 708 may be used to capture corresponding image data substantially concurrently, thus generating a UV image that is temporally aligned with a corresponding color image. Thus, the UV image generated by UV image sensor 708 may represent the same scene as the color image generated by color image sensor 706, and the UV image may thus pixel-wise correspond to the color image.

In some implementations, two instantiations of optical system 700 may be arranged in a predetermined pose relative to one another, thus forming a color-UV stereoscopic image capture apparatus. Specifically, the color-UV stereoscopic image capture apparatus may include two instances of objective lens 702, two instances of wavelength splitter 704, two instances of color image sensor 706, and two instances of UV image sensor 708. Accordingly, a (UV) disparity between (i) a first UV image captured using a first instance of UV image sensor 708 and (ii) a second UV image captured using a second instance of UV image sensor 708 may be used to determine a first depth image. Additionally or alternatively, a (color) disparity between (i) a first color image captured using a first instance of color image sensor 706 and (ii) a second color image captured using a second instance of color image sensor 706 may be used to determine the first depth image and/or a second depth image. Depth images generated using the color-UV stereoscopic image capture apparatus may be spatially and temporally aligned with corresponding color and/or UV images, thus providing another layer of information about the scene represented by the corresponding color and/or UV images.

### VI. EXAMPLE PROCESSING OPERATIONS OF UV IMAGES AND COLOR IMAGES

The UV images, color images, and/or depth images generated using any of the techniques and/or systems described above may be processed to identify therein one or more features of the environment (e.g., objects and/or characteristics thereof). In one example, the color, UV, and/or depth images may be processed using one or more rules-based and/or heuristic-based algorithms configured to detect, within these images, predetermined features, such as edges or blobs, among other possibilities. In another example, the color, UV, and/or depth images may be processed using one or more machine learning models/algorithms (e.g., artificial neural networks) that have been trained to identify, within these images, the one or more features of the environment.

In some implementations, the corresponding color, UV, and/or depth images may be processed independently. For example, a first UV-specific machine learning model may be configured to identify features within UV images, while a second color-specific machine learning model may be configured to identify features within color images. In other implementations, corresponding color, UV, and/or depth images may be processed jointly. For example, a model or algorithm may be configured to receive as input a concatenation of the UV, color, and/or depth images, and may be configured to identify the one or more features by jointly considering visual patterns present across multiple channels (e.g., R, G, B, UV, and/or depth) of the concatenation, rather than by considering each channel independently. When the corresponding color, UV, and/or depth images are accurately aligned (e.g., spatially and temporally), the resulting concatenation may also be accurately aligned, resulting in all channels of a given pixel of the concatenation representing attributes of a same portion of the environment, and thus allowing for more accurate identification of the one or more features.

These color, UV, and depth images may provide complementary information in that each type of image may represent features and/or aspects thereof that might not be represented in other types of images. For example, UV images may be used to identify portions of the environment to be cleaned based on the UV images representing stains that are not apparent in corresponding color images, while color images may facilitate performance of any tasks that depend on determination of the colors of different features. Additionally, some features of the environment may exhibit UV fluorescence. That is, some features may emit visible light in response to having UV light shined thereon. The fluorescence may be observable in the color images, but might not be observable in the corresponding UV images (although other aspects of the fluorescent features may be represented in the UV images). Accordingly, such fluorescent features may be easier to identify when the visible and UV light components are separately represented, and the two components are accurately aligned in time and space.

### VII. ADDITIONAL EXAMPLE OPERATIONS

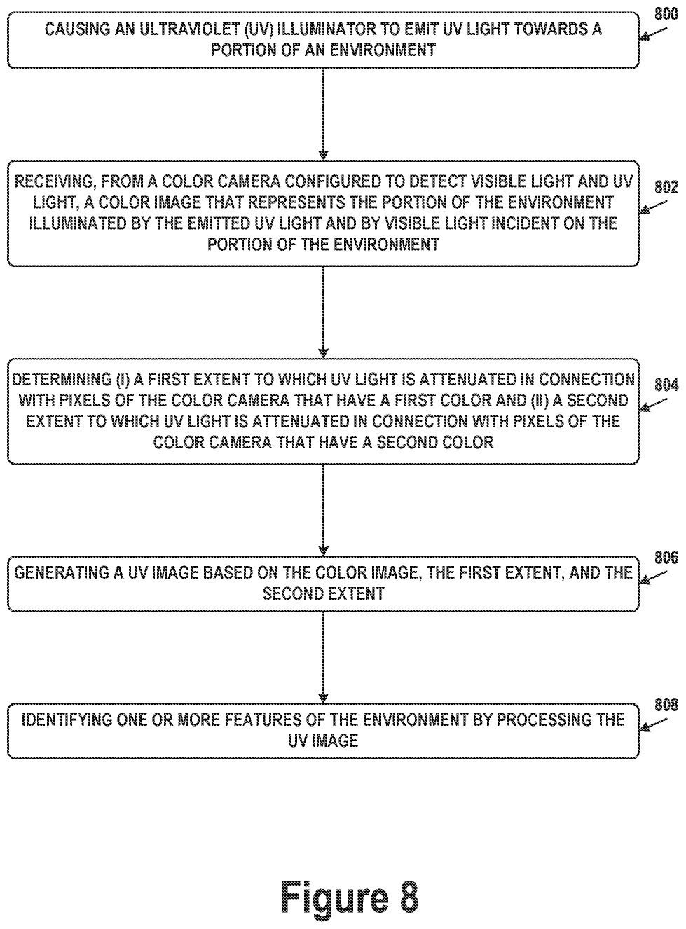

FIG. 8 illustrates a flow chart of operations related to generating a UV image and identifying one or more features of the environment based on the UV image. The operations may be carried out by robotic system 100, robot 200, system 620, and/or system 650, among other possibilities. The embodiments of FIG. 8 may be simplified by the removal of any one or more of the features shown therein. Further, these embodiments may be combined with features, aspects, and/or implementations of any of the previous figures or otherwise described herein.

Block 800 may involve causing a UV illuminator to emit UV light towards a portion of an environment.

Block 802 may involve receiving, from a color camera configured to detect visible light and UV light, a color image that represents the portion of the environment illuminated by the emitted UV light and by visible light incident on the portion of the environment.

Block 804 may involve determining (i) a first extent to which UV light is attenuated in connection with pixels of the color camera that have a first color and (ii) a second extent to which UV light is attenuated in connection with pixels of the color camera that have a second color.

Block 806 may involve generating a UV image based on the color image, the first extent, and the second extent.

Block 808 may involve identifying one or more features of the environment by processing the UV image.

In some embodiments, generating the UV image may include determining a weighting matrix based on the first extent and the second extent, and determining a color-UV image that includes, for each respective pixel of the color image, a corresponding pixel that includes a plurality of values. The plurality of values may include one or more color values representing a visible light component of the respective pixel and a UV value representing a UV light component of the respective pixel. Generating the UV image may also include determining a transformation loss value by determining, for each respective pixel of the color image, a difference between (i) one or more values of the respective pixel and (ii) corresponding products of the weighting matrix and the plurality of values of the corresponding pixel of the color-UV image. Generating the UV image may further include adjusting, for each of one or more pixels of the color-UV image, the plurality of values to reduce the transformation loss value below a threshold value.

In some embodiments, generating the UV image may include determining, using a regularization function, a regularization loss value based on the color-UV image, and adjusting, for each of the one or more pixels of the color-UV image, the plurality of values to reduce a sum of the transformation loss value and the regularization loss value to below the threshold value.

In some embodiments, the regularization function may be parametrized as a machine learning model. Determining the regularization loss value based on the color-UV image may include processing the color-UV image using the machine learning model. The regularization loss value may be based on an output of the machine learning model.

In some embodiments, for each respective pixel of the color image, the one or more values of the respective pixel may represent both the visible light component of the respective pixel and the UV light component of the respective pixel. For each respective pixel of the color image, the corresponding pixel in the color-UV image may represent the visible light component of the respective pixel using the one or more color values that are separate from the UV value used to represent the UV light component of the respective pixel.

In some embodiments, generating the UV image may include generating the UV image based on the UV value of each respective pixel of the color-UV image. An adjusted color image may be generated based on the one or more color values of each respective pixel of the color-UV image.

In some embodiments, the color image may be a first color image. Generating the UV image may include receiving, from the color camera, a second color image that represents the portion of the environment illuminated by additional visible light incident on the portion of the environment. Generating the UV image may also include determining a difference image based on the first color image and the second color image, and generating the UV image based on the difference image.

In some embodiments, generating the UV image may include adjusting (i) portions of the difference image corresponding to pixels having the first color based on the first extent and (ii) portions of the difference image corresponding to pixels having the second color based on the second extent.

In some embodiments, determining the first extent and the second extent may include determining a calibration UV image representing, for each respective pixel thereof, an extent to which the respective pixel of the color camera attenuates the UV light. Generating the UV image may further include adjusting, for each respective pixel of the difference image, a value of the respective pixel based on a value of a corresponding pixel of the calibration UV image.

In some embodiments, the calibration UV image may represent a scene (i) illuminated by UV light and (ii) expected, in the absence of attenuation of UV light by pixels of the color camera, to cause the pixels of the color camera to each generate substantially a maximum pixel value. Adjusting the value of the respective pixel may include dividing the value of the respective pixel by the value of the corresponding pixel of the calibration UV image.

In some embodiments, the one or more features of the environment may exhibit fluorescence in response to illumination by UV light. Generating the UV image may include demosaicing the difference image to generate a color representation of the one or more features of the environment exhibiting fluorescence. The one or more features of the environment may be identified based on the color representation, in the UV image, of the one or more features of the environment exhibiting fluorescence.

In some embodiments, identifying the one or more features of the environment by processing the UV image may include processing the UV image by a machine learning model that has been trained to identify the one or more features based on the UV image.

In some embodiments, processing the UV image by the machine learning model may include processing a concatenation of (i) the UV image and (ii) a color-only image by the machine learning model. The machine learning model may have been trained to identify the one or more features based on the concatenation.

In some embodiments, causing the UV illuminator to emit UV light towards the portion of the environment may include determining a distance between the portion of the environment and at least one of (i) the UV illuminator or (ii) the color camera, and selecting a power level with which to emit UV light based on the distance, the first extent, and the second extent.

In some embodiments, the UV image may be generated prior to demosaicing the color image.

In some embodiments, the one or more features of the environment (i) may be visually perceptible under illumination by the emitted UV light and (ii) might not be visually perceptible under illumination by visible light.

In some embodiments, the system used to generate the color and UV images may also include an image capture apparatus that includes (i) an objective lens configured to collect light reflected from the environment, (ii) a wavelength splitter configured to receive the light collected by the objective lens and separate the light collected by the objective lens into a UV light portion that is directed along a first optical path and a visible light portion that is directed along a second optical path, (iii) a UV image sensor situated along the first optical path and configured to measure the UV light portion, and (iv) a color image sensor situated along the second optical path and configured to measure the visible light portion. The UV illuminator may be caused to emit additional UV light towards a second portion of the environment. The UV image sensor may be caused to capture a second UV image that represents the second portion of the environment illuminated by the emitted additional UV light. The color image sensor may be caused to capture a second color image that represents the second portion of the environment illuminated by additional visible light incident on the second portion of the environment. The second UV image and the second color image may be captured substantially concurrently. One or more additional features of the environment may be identified by processing the second UV image and the second color image.

In some embodiments, the system may also include a second image capture apparatus that includes a second objective lens, a second wavelength splitter, a second UV image sensor, and a second color image sensor. A depth image of the second portion of the environment may be determined based on one or more of: (i) the second UV image and a third UV image captured by the second UV image sensor, or (ii) the second color image and a third color image captured by the second color image sensor. The one or more additional features of the environment may be identified by processing the depth image.

The systems and operations described herein with respect to UV images and color images may alternatively or additionally be applied in the context of IR and color images. That is, the systems and operations described herein may be used to generate IR images that are pixel-wise aligned with corresponding color images. Specifically, UV illuminator 500 may be replaced by an IR illuminator. Thus, UV light component 606 may instead be an IR light component, color attenuation values 626-628 may instead represent the attenuation of IR light by pixels of different colors, calibration image 636 may instead represent the attenuation of IR light by different pixels of the image sensor, adjusted UV light components 644 and 678 may instead represent adjusted IR light components, and weighting matrix 654 may be specific to IR light. Further, wavelength splitter 704 may be configured to direct visible light onto image sensor 706 and IR light onto image sensor 708. Other modifications may be made to adapt the optical path of a color image sensor to sense IR light instead of UV light.

### VIII. CONCLUSION

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. In the figures, similar symbols typically identify similar components, unless context dictates otherwise. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information may correspond to circuitry that can be configured to perform the specific logical functions of a herein-described method or technique. Alternatively or additionally, a block that represents a processing of information may correspond to a module, a segment, or a portion of program code (including related data). The program code may include one or more instructions executable by a processor for implementing specific logical operations or actions in the method or technique. The program code and/or related data may be stored on any type of computer readable medium such as a storage device including random access memory (RAM), a disk drive, a solid state drive, or another storage medium.

The computer readable medium may also include non-transitory computer readable media such as computer readable media that store data for short periods of time like register memory, processor cache, and RAM. The computer readable media may also include non-transitory computer readable media that store program code and/or data for longer periods of time. Thus, the computer readable media may include secondary or persistent long term storage, like read only memory (ROM), optical or magnetic disks, solid state drives, compact-disc read only memory (CD-ROM), for example. The computer readable media may also be any other volatile or non-volatile storage systems. A computer readable medium may be considered a computer readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A system comprising:
a color camera configured to detect visible light and ultraviolet (UV) light;
a UV illuminator;
a processor; and
a non-transitory computer-readable storage medium having stored thereon instructions that, when executed by the processor, cause the processor to perform operations comprising:
causing the UV illuminator to emit UV light towards a portion of an environment;
receiving, from the color camera, a color image that represents the portion of the environment illuminated by the emitted UV light and by visible light incident on the portion of the environment;
determining (i) a first extent to which UV light is attenuated in connection with pixels of the color camera that have a first color and (ii) a second extent to which UV light is attenuated in connection with pixels of the color camera that have a second color;
generating a UV image based on the color image, the first extent, and the second extent; and
identifying one or more features of the environment by processing the UV image.

2. The system of claim 1, wherein generating the UV image comprises:
determining a weighting matrix based on the first extent and the second extent;
determining a color-UV image that comprises, for each respective pixel of the color image, a corresponding pixel comprising a plurality of values, wherein the plurality of values comprises one or more color values representing a visible light component of the respective pixel and a UV value representing a UV light component of the respective pixel;
determining a transformation loss value by determining, for each respective pixel of the color image, a difference between (i) one or more values of the respective pixel and (ii) corresponding products of the weighting matrix and the plurality of values of the corresponding pixel of the color-UV image; and
adjusting, for each of one or more pixels of the color-UV image, the plurality of values to reduce the transformation loss value below a threshold value.

3. The system of claim 2, wherein generating the UV image comprises:
determining, using a regularization function, a regularization loss value based on the color-UV image; and
adjusting, for each of the one or more pixels of the color-UV image, the plurality of values to reduce a sum of the transformation loss value and the regularization loss value to below the threshold value.

4. The system of claim 3, wherein the regularization function is parametrized as a machine learning model, and wherein determining the regularization loss value based on the color-UV image comprises:
processing the color-UV image using the machine learning model, wherein the regularization loss value is based on an output of the machine learning model.

5. The system of claim 2, wherein, for each respective pixel of the color image, the one or more values of the respective pixel represent both the visible light component of the respective pixel and the UV light component of the respective pixel, and wherein, for each respective pixel of the color image, the corresponding pixel in the color-UV image represents the visible light component of the respective pixel using the one or more color values that are separate from the UV value used to represent the UV light component of the respective pixel.

6. The system of claim 2, wherein:
generating the UV image comprises generating the UV image based on the UV value of each respective pixel of the color-UV image; and
the operations further comprise generating an adjusted color image based on the one or more color values of each respective pixel of the color-UV image.

7. The system of claim 1, wherein the color image is a first color image, and wherein generating the UV image comprises:
receiving, from the color camera, a second color image that represents the portion of the environment illuminated by additional visible light incident on the portion of the environment;
determining a difference image based on the first color image and the second color image; and
generating the UV image based on the difference image.

8. The system of claim 7, wherein generating the UV image comprises:
adjusting (i) portions of the difference image corresponding to pixels having the first color based on the first extent and (ii) portions of the difference image corresponding to pixels having the second color based on the second extent.

9. The system of claim 7, wherein:
determining the first extent and the second extent comprises determining a calibration UV image representing, for each respective pixel thereof, an extent to which the respective pixel of the color camera attenuates the UV light; and
generating the UV image further comprises adjusting, for each respective pixel of the difference image, a value of the respective pixel based on a value of a corresponding pixel of the calibration UV image.

10. The system of claim 9, wherein:
the calibration UV image represents a scene (i) illuminated by UV light and (ii) expected, in the absence of attenuation of UV light by pixels of the color camera, to cause the pixels of the color camera to each generate substantially a maximum pixel value; and
adjusting the value of the respective pixel comprises dividing the value of the respective pixel by the value of the corresponding pixel of the calibration UV image.

11. The system of claim 7, wherein the one or more features of the environment exhibit fluorescence in response to illumination by UV light, and wherein generating the UV image comprises:
demosaicing the difference image to generate a color representation of the one or more features of the environment exhibiting fluorescence, wherein the one or more features of the environment are identified based on the color representation, in the UV image, of the one or more features of the environment exhibiting fluorescence.

12. The system of claim 1, wherein identifying the one or more features of the environment by processing the UV image comprises:
processing the UV image by a machine learning model that has been trained to identify the one or more features based on the UV image.

13. The system of claim 12, wherein processing the UV image by the machine learning model comprises:
processing a concatenation of (i) the UV image and (ii) a color-only image by the machine learning model, wherein the machine learning model has been trained to identify the one or more features based on the concatenation.

14. The system of claim 1, wherein causing the UV illuminator to emit UV light towards the portion of the environment comprises:
determining a distance between the portion of the environment and at least one of (i) the UV illuminator or (ii) the color camera; and
selecting a power level with which to emit UV light based on the distance, the first extent, and the second extent.

15. The system of claim 1, wherein the UV image is generated prior to demosaicing the color image.

16. The system of claim 1, wherein the one or more features of the environment (i) are visually perceptible under illumination by the emitted UV light and (ii) are not visually perceptible under illumination by visible light.

17. The system of claim 1, further comprising an image capture apparatus comprising:
an objective lens configured to collect light reflected from the environment;
a wavelength splitter configured to receive the light collected by the objective lens and separate the light collected by the objective lens into a UV light portion that is directed along a first optical path and a visible light portion that is directed along a second optical path;
a UV image sensor situated along the first optical path and configured to measure the UV light portion;
a color image sensor situated along the second optical path and configured to measure the visible light portion, wherein the operations further comprise:
causing the UV illuminator to emit additional UV light towards a second portion of the environment;
causing the UV image sensor to capture a second UV image that represents the second portion of the environment illuminated by the emitted additional UV light;
causing the color image sensor to capture a second color image that represents the second portion of the environment illuminated by additional visible light incident on the second portion of the environment, wherein the second UV image and the second color image are captured substantially concurrently; and
identifying one or more additional features of the environment by processing the second UV image and the second color image.

18. The system of claim 17, further comprising a second image capture apparatus comprising a second objective lens, a second wavelength splitter, a second UV image sensor, and a second color image sensor, wherein the operations further comprise:
determining a depth image of the second portion of the environment based on one or more of: (i) the second UV image and a third UV image captured by the second UV image sensor, or (ii) the second color image and a third color image captured by the second color image sensor; and
identifying the one or more additional features of the environment by processing the depth image.

19. A computer-implemented method comprising:
causing an ultraviolet (UV) illuminator to emit UV light towards a portion of an environment;
receiving, from a color camera configured to detect visible light and UV light, a color image that represents the portion of the environment illuminated by the emitted UV light and by visible light incident on the portion of the environment;
determining (i) a first extent to which UV light is attenuated in connection with pixels of the color camera that have a first color and (ii) a second extent to which UV light is attenuated in connection with pixels of the color camera that have a second color;
generating a UV image based on the color image, the first extent, and the second extent; and
identifying one or more features of the environment by processing the UV image.

20. A non-transitory computer-readable storage medium having stored thereon instructions that, when executed by a computing device, cause the computing device to perform operations comprising:
causing an ultraviolet (UV) illuminator to emit UV light towards a portion of an environment;
receiving, from a color camera configured to detect visible light and UV light, a color image that represents the portion of the environment illuminated by the emitted UV light and by visible light incident on the portion of the environment;
determining (i) a first extent to which UV light is attenuated in connection with pixels of the color camera that have a first color and (ii) a second extent to which UV light is attenuated in connection with pixels of the color camera that have a second color;
generating a UV image based on the color image, the first extent, and the second extent; and
identifying one or more features of the environment by processing the UV image.
